(19) United States
(12) Patent Application Publication
(10) Pub. No.: US 2026/0165611 A1
(43) Pub. Date: Jun. 18, 2026

(54) Systems and Methods for Monitoring Biometric Data

(71) Applicant: IDEXX Laboratories, Inc., Westbrook, ME (US)

(72) Inventor: Evan Peck, Mechanicsburg, PA (US)

(73) Assignee: IDEXX Laboratories, Inc., Westbrook, ME (US)

(21) Appl. No.: 19/303,687

(22) Filed: Aug. 19, 2025

Related U.S. Application Data
(60) Provisional application No. 63/734,316, filed on Dec. 16, 2024.

(51) Int. Cl. A61B 5/145; A61B 5/00; G16H 50/20; G16H 50/70

(52) U.S. Cl. CPC A61B 5/14514; A61B 5/14532; A61B 5/6815; G16H 50/20; G16H 50/70; A61B 2503/40; A61B 2562/0247

(57) ABSTRACT

A computer implemented method for monitoring biometric data from a non-human subject is disclosed. The method includes receiving data a from a device, the device comprising one or more sensors and one or more microneedles structurally configured to contact interstitial fluid of a non-human subject’s ear, wherein the data is indicative of one or more constituents within the interstitial fluid. The method also includes processing the data from the device to determine the one or more constituents within the interstitial fluid. The method further includes, based at least in part on the determined one or more constituents within the interstitial fluid and historic data from the device, determining one or more conditions of the non-human subject. The method additionally includes providing an indication of the one or more determined conditions of the non-human subject.

### CROSS-REFERENCE TO RELATED APPLICATIONS

This application claims the benefit of co-pending U.S. Provisional Patent Application Serial No. 63/734,316, filed December 16, 2024, which is hereby incorporated by reference in its entirety.

### FIELD OF THE DISCLOSURE

The present disclosure involves systems and methods for monitoring biological data from an animal. Namely, devices and methods of the disclosure receive data from a device, the device utilizing a microneedle array of one or more microneedles.

### BACKGROUND

Microneedles can be utilized for a variety of different tasks, including tasks relating to collecting a biological sample from an animal.

### SUMMARY

In particular, microneedles can be used to pierce the skin of an animal and transport biological sample from the animal to a collection device. Further, an array of microneedles can be utilized to simultaneously collect sample while reducing collection time without enhancing pain or discomfort experienced by the animal as compared to large, conventional needles. Additionally or alternatively, various sensors can be positioned at or near the tip of one or more microneedles to continuously or intermittently monitor the biological sample.

When sample is drawn from an animal to perform one or more tests (e.g., blood tests), often a blood draw is conducted at a particular location, such as a veterinary office. More particularly, intravenous blood draws with a conventional needle may require specialized training to locate and access appropriate veins of the animal, and may be performed by veterinarians or veterinary technicians at a veterinary office. However, animals are not always comfortable, responsive, and/or cooperative at unfamiliar locations (e.g., a veterinary office) and/or with one or more parties administering the blood draw (e.g., a veterinary technician or veterinarian). Also, conventional needles may draw large amounts of fluids (e.g., blood) from the animal to perform the tests. Accordingly, existing testing devices and procedures for animals can be difficult to execute, as well uncomfortable and invasive to the animal.

The present disclosure is directed to systems and methods for monitoring biometric data, particularly interstitial fluid, from a non-human subject utilizing a device (e.g., an ear tag) which includes microneedles and a sensor. Namely, a device, such as an ear tag, can monitor interstitial fluid from a non-human subject via the microneedles. A sensor on the device, in some cases on the microneedles, can collect data indicative of one or more constituents within the interstitial fluid. The collected data from the sensor can be transmitted to a computing device to determine one or more conditions of the subject, based on the determined one or more constituents within the interstitial fluid and historic data (e.g., data related to an interstitial fluid sample collected an earlier time).

Interstitial fluid is accessible closer to the surface of the skin than blood. If needed, the microneedles can remain in contact with a subject’s appendage (e.g., the ear) for an extended period of time to facilitate monitoring of biological data over time. Because the systems and methods of the present disclosure utilize microneedles, minimal training may be required for a user to position the device and contact interstitial fluid from the animal, and the device placement can be conducted by users familiar to the animal, such as the animal’s caretaker. Moreover, because the device placement can be conducted by users with minimal training, the device placement can be conducted in any suitable location, such as the animal’s home or other location familiar to the animal. Further, because interstitial fluid is more accessible closer to the surface of the skin than blood, contacting the interstitial fluid may be less invasive than a blood draw.

In an example, a computer implemented method for monitoring biometric data from a non-human subject is disclosed. The method includes receiving data from a device, the device comprising one or more sensors and one or more microneedles structurally configured to contact interstitial fluid of a non-human subject’s ear, wherein the data is indicative of one or more constituents within the interstitial fluid. The method also includes processing the data from the device to determine the one or more constituents within the interstitial fluid. The method further includes based at least in part on the determined one or more constituents within the interstitial fluid and historic data from the device, determining one or more conditions of the non-human subject. The method additionally includes providing an indication of the one or more determined conditions of the non-human subject.

In another example, a non-transitory computer-readable medium, having stored thereon program instructions that, upon execution by a processor, cause performance of a set of operations is disclosed. The set of operations includes receiving data from a device, the device comprising one or more sensors and one or more microneedles structurally configured to contact interstitial fluid of a non-human subject’s ear, wherein the data is indicative of one or more constituents within the interstitial fluid. The set of operations also includes processing the data from the device to determine the one or more constituents within the interstitial fluid. The set of operations further includes based at least in part on the determined one or more constituents within the interstitial fluid and historic data from the device, determining one or more conditions of the non-human subject. The set of operations additionally includes providing an indication of the one or more determined conditions of the non-human subject.

In another example, a computing system is disclosed, the computing system including a processor and a non-transitory computer-readable medium, having stored thereon program instructions that, upon execution by the processor, cause performance of a set of operations. The set of operations includes receiving data from a device, the device comprising one or more sensors and one or more microneedles structurally configured to contact interstitial fluid of a non-human subject’s ear, wherein the data is indicative of one or more constituents within the interstitial fluid. The set of operations also includes processing the data from the device to determine the one or more constituents within the interstitial fluid. The set of operations further includes based at least in part on the determined one or more constituents within the interstitial fluid and historic data from the device, determining one or more conditions of the non-human subject. The set of operations additionally includes providing an indication of the one or more determined conditions of the non-human subject.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The above, as well as additional, features will be better understood through the following illustrative and non-limiting detailed description of example embodiments, with reference to the appended drawings.

FIG. 1 illustrates a simplified block diagram of an example computing device, according to an example embodiment.

FIG. 2A illustrates an example device, according to an example embodiment.

FIG. 2B illustrates an example device, according to an example embodiment.

FIG. 2C illustrates an exploded view of a microneedle array, according to an example embodiment.

FIG. 2D illustrates an assembled view of the microneedle array of FIG. 2C, according to an example embodiment.

FIG. 2E illustrates an enlarged section view of the microneedle array of FIG. 2C, according to an example embodiment.

FIG. 2F illustrates an enlarged section view of a microneedle array, according to an example embodiment.

FIG. 2G illustrates a microneedle in isolation, according to an example embodiment.

FIG. 2H illustrates an exploded view of a microneedle array, according to an example embodiment.

FIG. 2I illustrates an assembled view of the microneedle array of FIG. 2F, according to an example embodiment.

FIG. 2J illustrates an assembled view of a microneedle array, according to an example embodiment.

FIG. 2K illustrates an enlarged section view of the microneedle array of FIG. 2H, according to an example embodiment.

FIG. 3 illustrates a device including a microneedle array, according to an example embodiment.

FIG. 4 illustrates a flowchart of a method, according to an example embodiment.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example.

When biological sample from an animal subject is drawn to be analyzed (e.g., for a blood test), often these draws are performed on the animal at a particular location and/or by a particular party (e.g., such as by bringing an animal to a veterinary office or bringing a travelling veterinarian to an animal). However, animals are not always comfortable, responsive, and/or cooperative in this process.

To help address these issues, embodiments of the present disclosure include analyzing data related to interstitial fluid of a subject. The interstitial fluid can be monitored utilizing a device, such as a device, which includes a microneedle array, the microneedle array including sensors, that can be utilized to contact, monitor, and/or collect a sample from an animal without being as offensive and destabilizing to the animal as the conventional sample collection methods. Interstitial fluid surrounds cells and tissues and can contain concentrations of biomarkers typically found in the blood, as well as biomarkers that can be detected based on metabolic activity in the body. Interstitial fluid may also be accessed closer to the skin than blood. As such, the penetration depth of the needle may be much smaller than in traditional blood collection devices. Further, continuous or intermittent monitoring can provide a more fulsome indication of the animal’s health. For instance, it can help detect changes in vital signs and potentially detect early signs of various diseases.

Additionally, in example implementations, the device is an ear tag. Namely, the device adheres to the underside of the ear, which typically has very little fur and thinner skin. As such, interstitial fluid may be accessed, monitored, and collected very close to the surface of the skin on the underside of the ear. Thus, shorter lengths of each microneedle may be utilized.

The sensor is in communication with a computing device. In this manner, the sensor on the device can collect data indicative of one or more constituents within the interstitial fluid. The collected data from the sensor can be transmitted to a computing device to determine one or more conditions of the subject, based on the determined one or more constituents within the interstitial fluid and historic data (e.g., data related to an interstitial fluid sample collected an earlier time). Accordingly, the biometric data of the subject may be monitored over time.

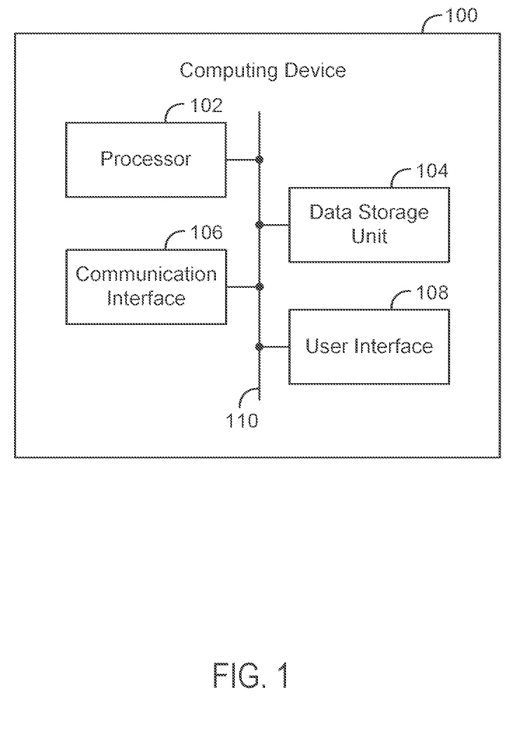

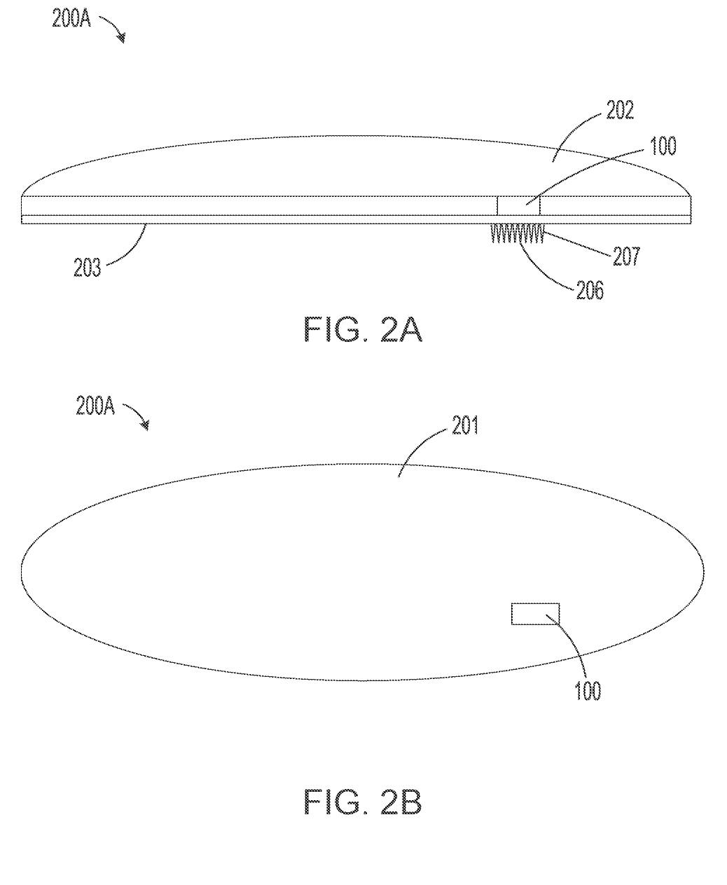

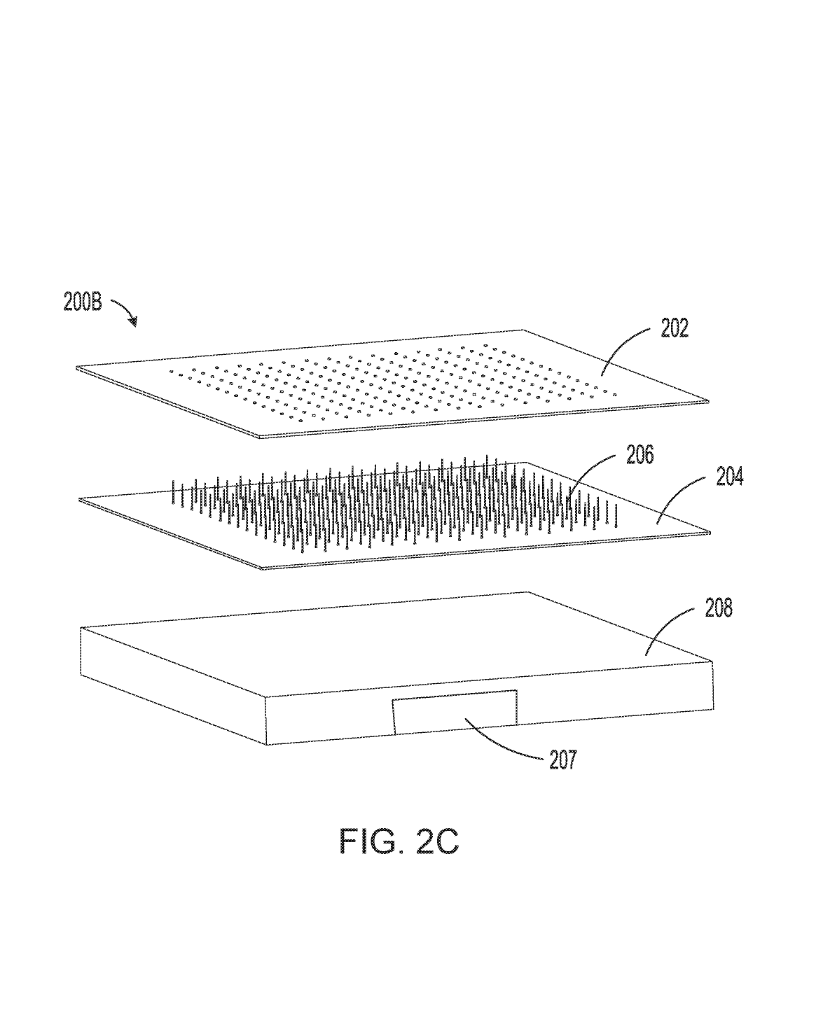

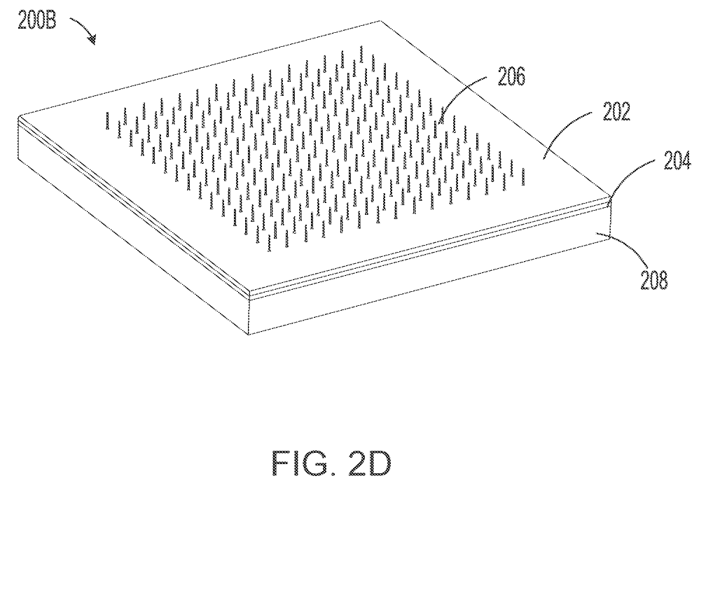

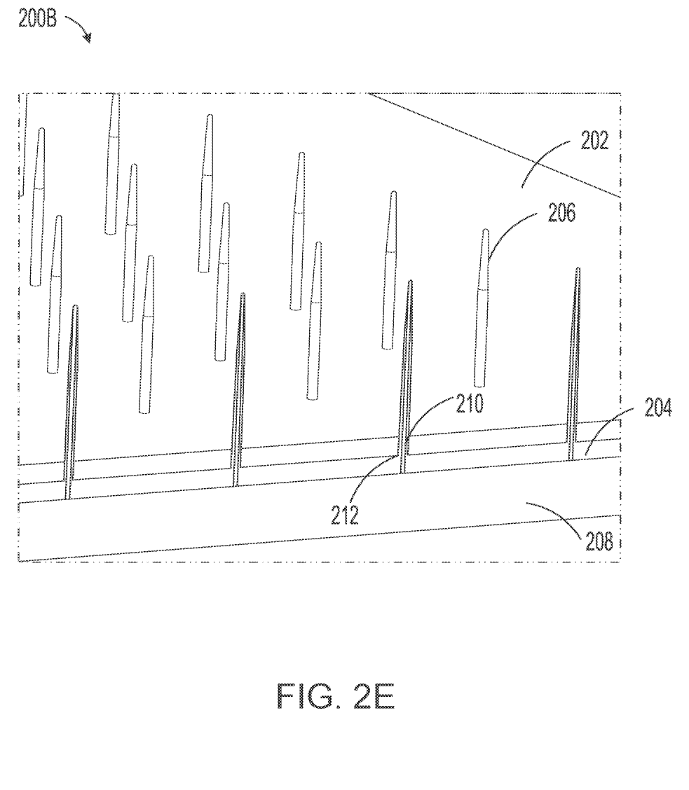

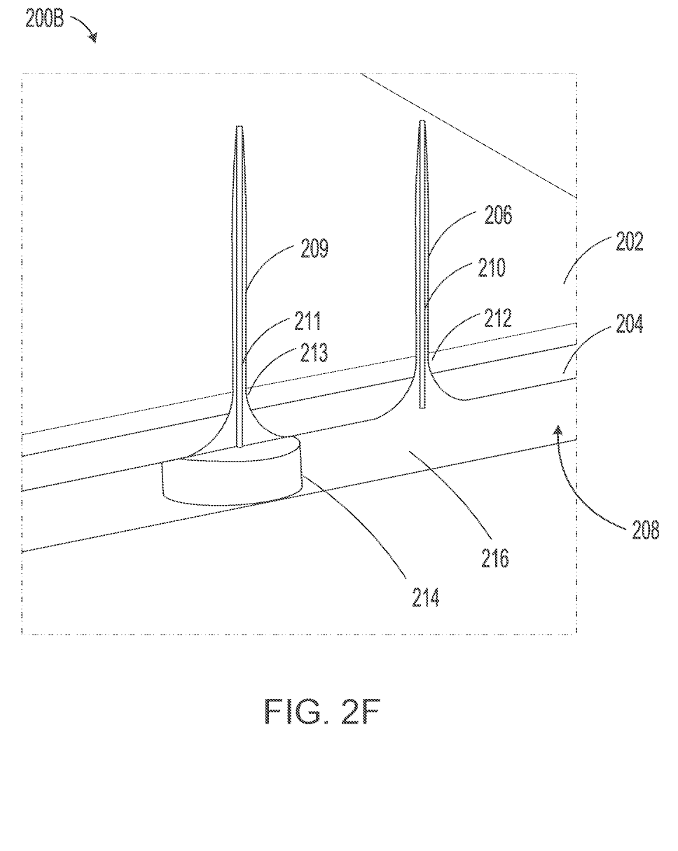

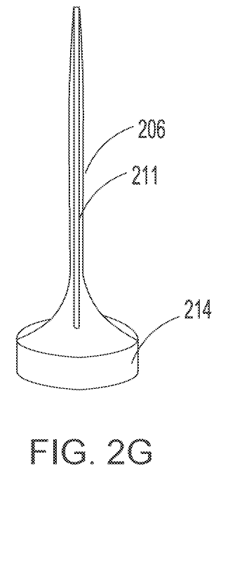

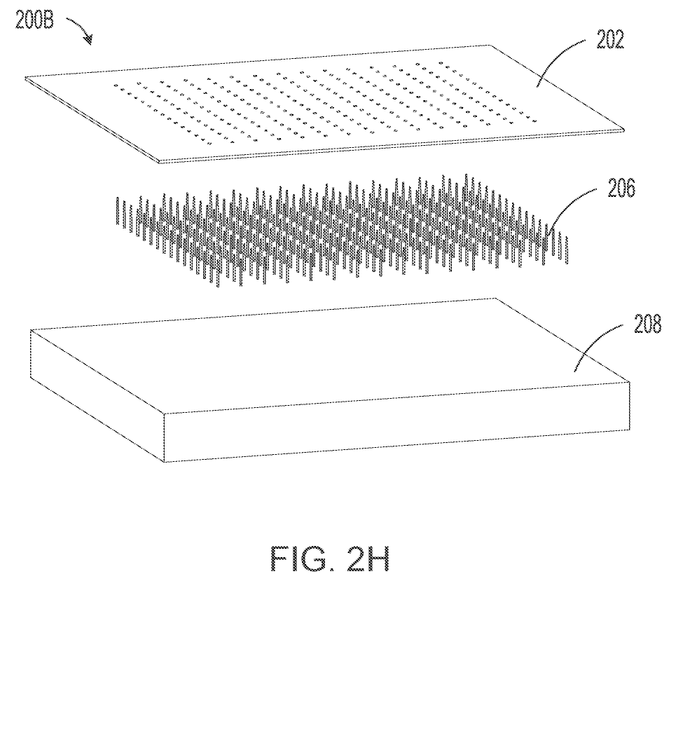

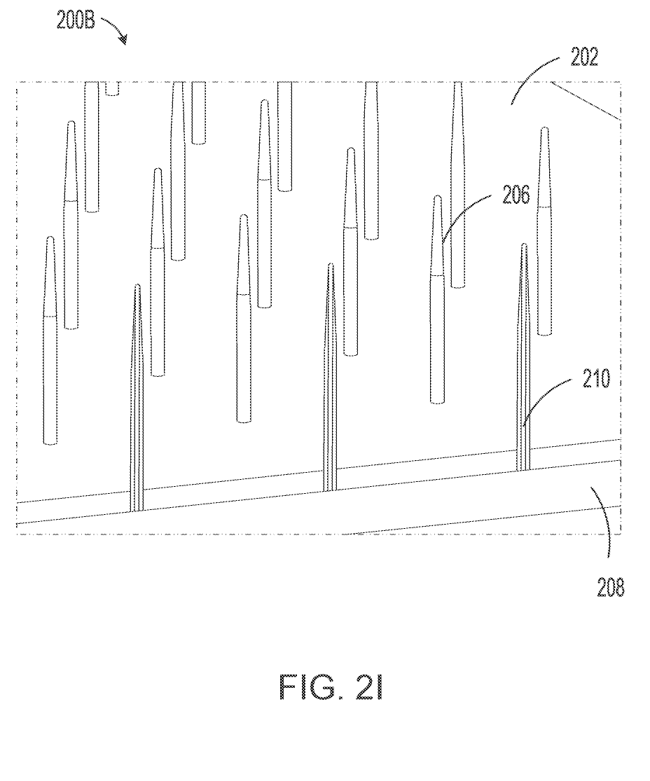

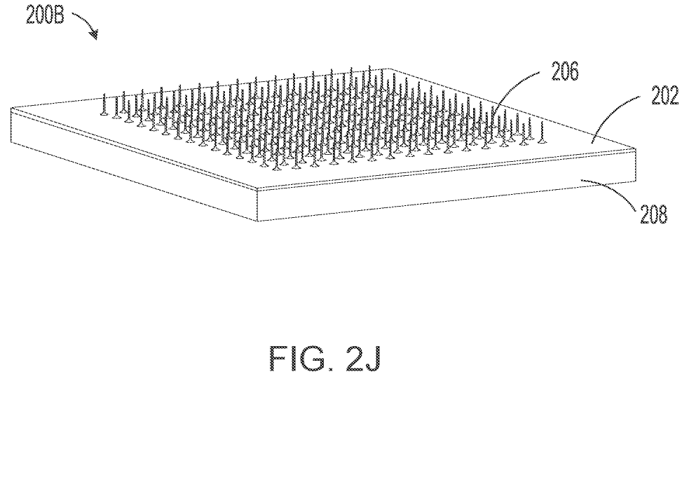

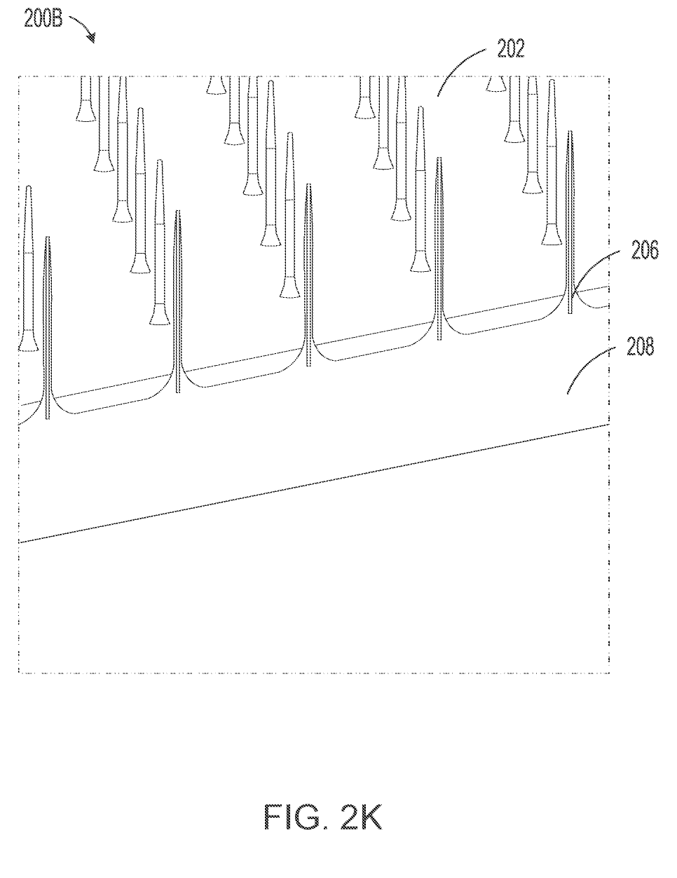

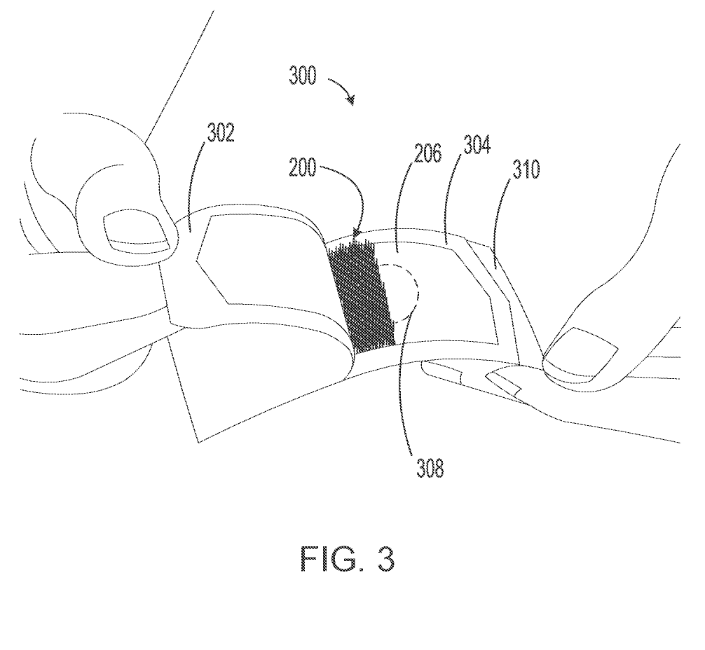

Referring now to the figures, FIG. 1 is a simplified block diagram of an example computing device 100 of a system (e.g., that can be utilized with devices and methods illustrated in FIGS. 2-3, described in further detail below). Computing device 100 can perform various acts and/or functions, such as those described in this disclosure. Computing device 100 can include various components, such as processor 102, data storage unit 104, communication interface 106, and/or user interface 108. These components can be connected to each other (or to another device, system, or other entity) via connection mechanism 110.

Processor 102 can include a general-purpose processor (e.g., a microprocessor and/or a central processing unit (CPU)) and/or a special-purpose processor (e.g., a digital signal processor (DSP) and/or a graphics processing unit (GPU)).

Data storage unit 104 can include one or more volatile, non-volatile, removable, and/or non-removable storage components, such as magnetic, optical, or flash storage, and/or can be integrated in whole or in part with processor 102. Further, data storage unit 104 can take the form of a non-transitory computer-readable storage medium, having stored thereon program instructions (e.g., compiled or non-compiled program logic and/or machine code) that, when executed by processor 102, cause computing device 100 to perform one or more acts and/or functions, such as those described in this disclosure. As such, computing device 100 can be configured to perform one or more acts and/or functions, such as those described in this disclosure. Such program instructions can define and/or be part of a discrete software application. In some instances, computing device 100 can execute program instructions in response to receiving an input, such as from communication interface 106 and/or user interface 108. Data storage unit 104 can also store other types of data, such as those types described in this disclosure.

Communication interface 106 can allow computing device 100 to connect to and/or communicate with another other entity according to one or more protocols. In one example, communication interface 106 can be a wired interface, such as an Ethernet interface or a high-definition serial-digital-interface (HD-SDI). In another example, communication interface 106 can be a wireless interface, such as a cellular or WI FI interface. In this disclosure, a connection can be a direct connection or an indirect connection, the latter being a connection that passes through and/or traverses one or more entities, such as a router, switcher, or other network device. Likewise, in this disclosure, a transmission can be a direct transmission or an indirect transmission.

User interface 108 can facilitate interaction between computing device 100 and a user of computing device 100, if applicable. As such, user interface 108 can include input components such as a keyboard, a keypad, a mouse, a touch sensitive panel, a microphone, a camera, and/or a movement sensor, all of which can be used to obtain data indicative of an environment of computing device 100, and/or output components such as a display device (which, for example, can be combined with a touch sensitive panel), a sound speaker, and/or a haptic feedback system. More generally, user interface 108 can include hardware and/or software components that facilitate interaction between computing device 100 and the user of the computing device 100.

Computing device 100 can take various forms, such as a workstation terminal, a desktop computer, a laptop, a tablet, a mobile phone, or a controller.

Referring now to FIGS. 2A-2I, which illustrate example devices 200A and 200B. More particularly, FIGS. 2A illustrates a cross-sectional view of an example device 200A for monitoring a biological sample. FIG. 2B illustrates a top view of example device 200A for monitoring biological sample. FIG. 2C illustrates an exploded view of example device 200B, FIG. 2D illustrates an assembled view of the device 200B of FIG. 2C, and FIG. 2E illustrates an enlarged section view of the microneedle array of FIG. 2C. FIG. 2F illustrates an enlarged section view of a microneedle array of another example device 200B. FIG. 2G illustrates a microneedle in isolation that could be used in accordance with the example devices 200A and/or 200B. FIG. 2H illustrates an exploded view of another example device 200A and/or 200B, and FIG. 2I illustrates an assembled view of the device 200B of FIG. 2H. FIG. 2J illustrates an assembled section view of another example device 200B, and FIG. 2K illustrates an enlarged section view of the microneedle array of FIG. 2J.

As shown in FIGS. 2A-2B, the device 200A includes a backing 201, an adhesive layer 203, a microneedle array 206, and a computing device 100. In example implementations one or more of the microneedles in the microneedle array 206 include one or more sensors 207. In some examples, the microneedles can include a variety of types of sensors. Additionally or alternatively, in some embodiments, several microneedles can include the same type of sensor. The one or more sensors 207 are configured to contact a biological fluid (e.g., interstitial fluid and/or blood) to provide continuous and/or intermittent monitoring.

In examples, the backing 201 can include flexible materials, such as woven fabric, elastic rubber, elastic yarn, or polymers, for instance. Many example materials are possible. The flexibility of the backing 201 facilitates placement of the device 200A on the subject over an extended period of time. Namely, as the subject moves, the backing 201 is configured flex and bend, as necessary, while still remaining adhered to the subject.

In example implementations, the device 200A includes an adhesive layer 203 to adhere the device 200A to an appendage (e.g., the ear) of the subject. In example implementations the adhesive can help adhere the device 200A to the appendage of the subject over a period of time (e.g., hours, days, or weeks) to allow the one or more sensors 207 and/or computing device 100 to collect data over a period of time. In some examples, the adhesive material is distributed throughout the entire outer surface of the backing 201. In other examples, the adhesive material may be on certain areas (e.g., the outer perimeter surrounding the microneedle array 206) of the outer surface of the backing 201. As noted above, the device 200A may be an ear tag. The adhesive on the backing 201 can include strong adhesives to allow the ear tag to remain adhered to the subject’s ear for an extended period of time. In example implementations, the adhesive can include materials such as vinyl resins, acrylates, methacrylates, epoxy diacrylates, acrylic polymer, among other possibilities. Further, the adhesive can be hypoallergenic to prevent irritation.

The device 200A includes a microneedle array 206. The microneedle array 206 is positioned on the same side of the backing 201 as the adhesive layer 203. In this manner, the microneedle array 206 is configured to puncture the surface of the skin of the subject to contact the interstitial fluid and/or blood of the subject. In the example shown in FIGS. 2A-2B, the microneedles of the microneedle array 206 are arranged in a grid (i.e., in aligned rows and columns), however, it should be understood that the microneedles of the microneedle array 206 can be arranged in any suitable formation.

In example embodiments, the shape and dimensions of each microneedle of the microneedle array can vary, depending on different uses. For example, longer lengths of each microneedle may be utilized for animals with larger appendages, thicker skin, or denser fur. Other examples are possible. In examples where the interstitial fluid sample is monitored from the underside of the ear, which typically has very little fur and thinner skin, the interstitial fluid is very close to the surface of the skin. Additionally, the underside of the ear may be a sensitive area for the subject. As such, in these examples, shorter lengths of each microneedle may be utilized.

In embodiments, the one or more microneedles may have one or more shapes, for example and without limitation: a cylindrical shape, a conical shape, a frustoconical shape, a pyramid shape, a square prism, a pentagonal prism, a hexagonal prism, an octagonal prism, and/or an annular prism, and/or any suitable combination thereof, including, for example, a microneedle that has a cylindrical base and terminates in a conical tip. Additionally or alternatively, in other examples, the microneedle array can be ergonomically shaped to better adhere to an animal’s ear. Other shapes are also possible.

In some example embodiments, the device 200A includes one or more additional substances positioned thereon and/or integrated therein. In some examples, adhesive layer 203 and/or the microneedles in microneedle array 206 includes a pharmaceutical agent positioned on and/or integrated within one or more microneedles of the microneedle array 206 and/or the adhesive layer 203, and that pharmaceutical agent may be transferred to the animal upon use. In some examples, this pharmaceutical agent may include an anesthetic to make the process more comfortable for the animal as compared to sample collection processes that do not include application of an anesthetic, among other possibilities. In some embodiments, the pharmaceutical agent may include an antibacterial substance or other agent to reduce the risk of infection and/or promote healing of the animal’s skin. In some examples, the pharmaceutical agent may include one or more substances or other agents to impart one or more pharmacological benefits to the animal during the sample collection process (e.g., treating the animal with a steroid medication, heartworm medication, etc.).

In example implementations, the microneedle array 206 includes one or more sensors 207. In some example configurations, the one or more sensors 207 may be at or near the end and/or tip of the microneedle so that the sensor penetrates far enough into the subject to make contact with the interstitial fluid and/or blood.

In example implementations, the one or more sensors 207 are configured to collect data indicative of one or more constituents within the sample (e.g., interstitial fluid). In some examples, the sensor 207 can include a pressure sensor to detect, for example, the interstitial pressure. Additionally or alternatively, in some examples, the sensor 207 can include a glucose sensor to detect a glucose level of the collected sample. Additionally or alternatively, in some examples, the sensor 207 can include a salinity sensor to detect a salinity level of the collected sample. In some example implementations, the one or more sensors 207 can collect data to determine the presence of one or more biomarkers in the sample, including, but not limited to hormones, electrolytes, peptides, enzymes, proteins, or antibodies, and others. Further, in some example implementations, the one or more sensors 207 can collect data to monitor drug concentrations within the body of the subject. In some examples, the device 200A can include a combination of different types of sensors (e.g., a pressure sensor and a glucose sensor). Many example types of sensors and combinations of sensors are possible.

In example embodiments, the sensor 207 can collect data over different periods of time, depending on different uses. For instance some example implementations, the sensor 207 takes an instantaneous measurement of the sample. In other examples, the sensor 207 may collect data over a configurable period of time. For instance, the subject may have the device 200A adhered to an appendage, such as an ear, over a period of minutes, hours or days. In these examples, the sensor 207 can collect data to determine if there is a change over time. For instance, in examples where the sensor 207 includes a glucose sensor, the sensor 207 can collect data over a number of hours or days. Many examples are possible.

In example implementations, the sensor 207 is in communication with computing device 100. In some example implementations, the computing device is positioned on the device 200A. In other example embodiments, the computing device may be separate from the device 200A. Additionally or alternatively, there may be a computing device 100 on device 200A in communication with a computing device separate from the device 200A (e.g., an external computer, a smartphone, etc.) For instance, the communication interface 106 can allow the computing device 100 to connect to and/or communicate with another the sensor 207 according to one or more protocols. In some examples the device 200A includes a computing device 100 which is in communication with an external computing device, similar to computing device 100, according to one or more protocols.

The computing device, whether attached to the device 200A or separate from it, receives data from the one or more sensors 207 of the device 200A. In example implementations, the computing device 100 can process the data collected from the one or more sensors 207 to determine one or more constituents within the collected sample. For instance, in examples where the collected sample includes interstitial fluid, the computing device 100 can process the data received from the sensor 207 to determine, for example, an interstitial pressure, a salinity level, and/or a glucose level, among other possibilities. Additionally or alternatively, the computing device can process the data received from the sensor 207 to determine the presence of one or more biomarkers in the sample, including, but not limited to hormones, electrolytes, peptides, enzymes, proteins, or antibodies, and others.

The computing device 100 can process the data received from the one or more sensors 207 to determine one or more conditions of the subject. In examples, this can include determining whether there were any changes over time and/or patterns, for example, by comparing one or more historic collected data points to more recent collected data. In examples, this can involve comparing instantaneous measurements taken at different times and/or analyzing data continuously collected over a period of time.

For instance, to compare two or more instantaneous measurements, the computing device can compare historic data (i.e., data collected at an earlier time) to more recent data collected. In examples where the one or more sensors 207 include a pressure sensor, the computing device can compare the interstitial pressure in the historic data from the one or more sensors 207 and determine if there is a change in the interstitial pressure based on the processed data.

Similarly, in examples where the one or more sensors 207 collect data continuously over a period of time, the computing device can process the historical data collected at an earlier time to more recent collected data to determine changes over time. In examples where the one or more sensors 207 include a glucose sensor and the glucose levels were collected over a number of hours or days to determine, the computing device can determine the changes in glucose levels over time. This can help determine, for instance, whether a subject has diabetes. In another example, this can help determine how the subject responds to certain food or drugs, for example. According to another example embodiment, in examples where the one or more sensors 207 collects data related to drug concentrations in a subject’s body, the computing device can process the data to determine drug dosage recommendations. Many example implementations are possible.

Although the example implementations shown in FIGS. 2A-2B utilize one or more microneedles, in some examples device 200A additionally or alternatively can include a different piercing mechanism. For instance, some in example embodiments the device 200A may include a lancet, a needle, and/or a prick configured to pierce the skin of a subject and monitor and/or draw a biological sample (such as interstitial fluid). Many examples are possible.

FIGS. 2C-2E illustrate another example device 200B, which includes an outer layer 202, an intermediate layer 204 comprising a microneedle array 206, and sample storage layer 208. In some embodiments and in the embodiment depicted in FIGS. 2C-2E, the microneedles of the microneedle array 206 are arranged in a grid (i.e., in aligned rows and columns), however, it should be understood that the microneedles of the microneedle array 206 can be arranged in any suitable formation. Additionally, in some example embodiments, the example device 200B can include one or more sensors 207 in communication with the sample storage layer 208. Further, the one or more sensors 207 include or are in communication with a computing device (such as computing device 100). The computing device receives information from the one or more sensors to determine one or more constituents within the collected sample and determine one or more conditions of the subject based on the processed data.

As seen in FIG. 2E, in some embodiments, one or more microneedles of the microneedle array 206 define the hollow inner channel 210, through which sample can pass from the animal to the sample storage layer 208. In some embodiments, the hollow inner channel 210 extends through the one or more microneedles of the microneedle array 206 and through the intermediate layer 204. Sample storage layer 208, in such embodiments, includes an absorbent material configured to receive sample from the hollow inner channel 210 of one or more microneedles in microneedle array 206 and the intermediate layer 204 and retain it until the sample is extracted from the device. Other examples are possible.

Sample fluid from the animal may also pass to the sample storage layer 208 along an outer surface of the one or more microneedles of the microneedle array 206. In some embodiments, one or more of the microneedles of the microneedle array 206 are solid and do not define the hollow inner channel 210. In these embodiments, sample fluid from the animal can be transported to the sample storage layer 208 along the surface of the one or more microneedles of the microneedle array 206. In some embodiments, the intermediate layer 204 is permeable or semi-permeable, such that sample fluid from the animal can pass through the intermediate layer 204. For example, in embodiments in which one or more microneedles of the microneedle array 206 are solid (i.e., do not define the channel 210), sample fluid from the animal may pass along the outer surface of the one or more microneedles of the microneedle array 206, through the intermediate layer 204, to the sample storage layer 208.

In some examples, the device 200B is arranged to extract an interstitial fluid sample from one or more appendages of an animal, such as the ear. In example embodiments, the device 200B includes at least one microneedle in the microneedle array 206 to pierce the skin of the animal and to collect an interstitial fluid sample. In some embodiments, the device 200B includes plurality of microneedles in the microneedle array 206 to pierce the skin of the animal and collect an interstitial fluid sample.

For example, individual microneedles of the microneedle array 206 may have a small diameter. In some embodiments, individual microneedles of the microneedle array 206 have a diameter less than about 1.0 millimeters (mm), less than about 0.5 mm, less than about 0.1 mm, less than about 50 micrometers (μm), less than about 25 μm, or the like. Furthermore, in some embodiments and as depicted in FIG. 2E, one or more of the microneedles of the microneedle array 206 define a hollow inner channel 210 through which interstitial fluid passes from the animal to the sample storage layer 208.

In example embodiments, the shape and dimensions of each microneedle of the microneedle array can vary, depending on different uses. For example, longer lengths of each microneedle may be utilized for animals with larger appendages, thicker skin, or denser fur. Other examples are possible. In examples where the interstitial fluid sample is monitored from the underside of the ear, which typically has very little fur and thinner skin, the interstitial fluid is very close to the surface of the skin. Additionally, the underside of the ear may be a sensitive area for the subject. As such, in these examples, shorter lengths of each microneedle may be utilized.

In embodiments, the one or more microneedles may have one or more shapes, for example and without limitation: a cylindrical shape, a conical shape, a frustoconical shape, a pyramid shape, a square prism, a pentagonal prism, a hexagonal prism, an octagonal prism, and/or an annular prism, and/or any suitable combination thereof, including, for example, a microneedle that has a cylindrical base and terminates in a conical tip. Additionally or alternatively, in other examples, the microneedle array can be ergonomically shaped to better adhere to an animal’s ear. Other shapes are also possible.

In one aspect, the at least one microneedle in the microneedle array 206 is in communication with the sample storage layer 208. For example, the at least one microneedle of the microneedle array 206 facilitates the transport of interstitial fluid from the animal to the sample storage layer 208. In some embodiments, the microneedle array 206 is monolithic with the intermediate layer 204. In some embodiments, the microneedle array 206 is coupled to the intermediate layer 204. In embodiments, the microneedle array 206 and the intermediate layer 204 are formed of the same material or from different materials. In some examples, the microneedles in the microneedle array may comprise a silicon needle weaved into an absorbent material, like a cotton pad, of the sample collection mechanism.

In example embodiments, the shape and dimensions of different microneedles of the microneedle array can vary, depending on different uses, including within a singular microneedle of microneedle array 206. In some examples, the microneedle array 206 may be altered and/or interchanged in a variety of ways. For example, one or more microneedles in the microneedle array 206 may be added or removed from the device and/or interchanged with one or more microneedles of a different configuration, shape, etc., among other possibilities. In any event, the at least one microneedle in the microneedle array 206 may transport fluid from the animal to the sample storage layer 208

In some embodiments, a user of the device 200B may apply pressure to the appendage of the animal with the device 200B until one or more designated events occur indicating a proper volume of sample has been collected to perform a test and/or the one or more sensors 207 has collected data. In some cases, the designated event might be the passage of a predetermined amount of time. For instance, the length of time to collect a predetermined volume of interstitial fluid, for example, for the one or more sensors 207 to collect data is between about 30 seconds and about 5 minutes, inclusive of the endpoints. In other examples, the device 200B is left on for an extended period of time to allow the one or more sensors 207 to continuously collect data over a period of time (e.g., hours, days, and/or weeks). In some embodiments, the sample storage layer 208 is structurally configured to change color when the predetermined volume of sample has been collected, for example as the result of saturation of the sample storage layer 208 and/or via interaction with the sample storage layer 208.

In example embodiments, the sample storage layer 208 is structurally configured to hold the collected sample until the sample is extracted from the sample storage layer 208. In some examples, the sample storage layer 208 is formed of or includes absorbent material that is designed to store samples, which may adhere and/or otherwise dry on the absorbent material. For example, in some embodiments, the sample storage layer 208 may include cotton, cellulose-based materials, filter paper, non-fibrous materials, or collection cards, which are typically absorbent and inert fibrous thin sheet materials, and the like and/or any suitable combination thereof. In some examples, the sample storage layer 208 define one or more chambers to store samples.

In some examples, such as depicted in FIG. 2F, a cross-section of an example device 200B is shown, according to an example embodiment. In this example embodiment, the device includes the outer layer 202, the intermediate layer 204 comprising the microneedle array comprising microneedles 206 and 209, and the sample storage layer 208 that includes a combination of an absorbent material 216 and defines one or more chambers 214 to store samples, among other possibilities. In example embodiments, if the sample storage layer 208 includes and/or is formed of an absorbent material and defines the at least one chamber, at least two different types of samples may be extracted from the sample storage layer 208 and analyzed: (i) a dried sample from the absorbent material portion 216 of the sample storage layer 208; and (ii) a fluid sample from the at least one chamber 214 of the sample storage layer 208. Other examples are possible.

In some examples, as illustrated in FIG. 2F, the sample may be transported: (1) through aperture 212 of outer layer 202 and intermediate layer 204 via hollow inner channel 210 of a microneedle of microneedle array 206 of the device 200 to the absorbent material portion 216 of the sample storage layer 208 and (2) through aperture 213 of outer layer 202 and intermediate layer 204 via channel 211 of microneedle 209 of device 200B to sample storage chamber portion 214 of sample storage layer 208. In some examples, sample can also pass through or along a microneedle of microneedle array 206 to the sample storage layer 208 itself, or both. While in the embodiment depicted in FIG. 2F, the sample storage chamber 214 is shaped as a curvette, it should be understood that the sample storage chamber 214 may have any suitable shape for storing fluid sample. Further, while a single storage chamber 214 is depicted in FIG. 2F, it should be understood that the device 200B can include any suitable number of storage chambers 214 associated with one or more of the microneedles of the microneedle array 206.

FIG. 2G depicts a singular microneedle 209 (e.g., as illustrated in FIG. 2F of device 200B) with channel 211, designed to drain into sample storage chamber 214. In some examples, sample storage chamber 214 may be designed to store a sample until the sample need be accessed for analysis and/or testing at a later time. The singular microneedle of the device 200B as depicted in FIG. 2G may be configured to be included in any embodiment in any embodiment of device 200B.

FIGS. 2H-2I illustrate another embodiment of the device 200B. Specifically, FIG. 2H illustrates an exploded view of example device 200B, FIG. 2I illustrates an assembled view of example device 200B of FIG. 2F.

As is shown in FIGS. 2H-2I and similar to the embodiment described above and depicted in FIGS. 2C-2E, in this example embodiment, the device 200B includes an outer layer 202, the microneedles in the microneedle array 206, and the sample storage layer 208. However, in the embodiment depicted in FIGS. 2H-2I, the device 200B does not include the intermediate layer 204 (e.g., as illustrated in FIG. 2A-2C).

FIGS. 2J-2K illustrate another embodiment of the device 200B. Specifically, FIG. 2J illustrates an assembled view of example device 200B, and FIG. 2K illustrates an enlarged section view of example device 200B of FIG. 2J.

As is shown in FIGS. 2J-2K and similar to the embodiment described above and depicted in FIGS. 2C-2E, in this example embodiment, the device 200B includes an outer layer 202, the microneedles in the microneedle array 206, and the sample storage layer 208. However, in the embodiment depicted in FIGS. 2J-2K, the device 200B does not include the intermediate layer 204 (e.g., as illustrated in FIG. 2C-2E) and the microneedles in the microneedle array 206 are assembled into the sample storage layer 208 (i.e., the microneedle array 206 is monolithic with the sample storage layer 208).

Although the example implementations shown in FIGS. 2C-2K utilize one or more microneedles, in some examples device 200B additionally or alternatively can include a different piercing mechanism. For instance, some in example embodiments the device 200B may include a lancet, a needle, and/or a prick configured to pierce the skin of a subject and monitor and/or draw a biological sample (such as interstitial fluid). Many examples are possible.

In example implementations, the sample storage layer 208 (and/or the one or more chambers 214 in example embodiments shown in FIGS. 2F-2G) is in communication with a sensor 207. The sensor 207 is configured to collect data indicative of one or more constituents within the of the collected sample (e.g., interstitial fluid). In some examples, the sensor 207 can include a pressure sensor to detect, for example, the interstitial pressure. Additionally or alternatively, in some examples, the sensor 207 can include a glucose sensor to detect a glucose level of the collected sample. Additionally or alternatively, in some examples, the sensor 207 can include a salinity sensor to detect a salinity level of the collected sample. In some example implementations, the one or more sensors 207 can collected data to determine the presence of one or more biomarkers in the sample, including, but not limited to hormones, electrolytes, peptides, enzymes, proteins, or antibodies, and others. Further, in some example implementations, the one or more sensors 207 can collect data to monitor drug concentrations within the body of the subject. In some examples, the device 200B can include a combination of different types of sensors (e.g., a pressure sensor and a glucose sensor). Many example types of sensors and combinations of sensors are possible.

In example embodiments, the sensor 207 can collect data over different periods of time, depending on different uses. For instance some example implementations, the sensor 207 takes an instantaneous measurement of the collected sample. In other examples, the sensor 207 may collect data over a configurable period of time. For instance, the subject may have the device 200B adhered to an appendage, such as an ear, over a period of minutes, hours or days. In these examples, the sensor 207 can collect data to determine if there is a change over time. For instance, in examples where the sensor 207 includes a glucose sensor, the sensor 207 can collect data over a number of hours or days. Many examples are possible.

In example implementations, the sensor 207 is in communication with a computing device, such as computing device 100. In some example implementations, the computing device is positioned on the device 200B. In other example embodiments, the computing device may be separate from the device 200B. For instance, the communication interface 106 can allow the computing device 100 to connect to and/or communicate with another the sensor 207 according to one or more protocols. In some examples the device 200B includes a computing device 100, similar to computing device 100, which is in communication with an external computing device, similar to computing device 100, according to one or more protocols.

The computing device, whether attached to the device 200B or separate from it, receives data from the sensor 207 of the device 200B. In example implementations, the computing device (e.g., computing device 100) can process the data collected from the one or more sensors 207 to determine one or more constituents within the collected sample. For instance, in examples where the collected sample includes interstitial fluid, the computing device 100 can process the data received from the sensor 207 to determine, for example, an interstitial pressure, a salinity level, and/or a glucose level, among other possibilities. Additionally or alternatively, the computing device can process the data received from the sensor 207 to determine the presence of one or more biomarkers in the sample, including, but not limited to hormones, electrolytes, peptides, enzymes, proteins, or antibodies, and others.

The computing device can process the data received from the one or more sensors 207 to determine one or more conditions of the subject. In examples, this can include determining whether there were any changes over time and/or patterns, for example, by comparing one or more historic collected data points to more recent collected data. In examples, this can involve comparing instantaneous measurements taken at different times and/or analyzing data continuously collected over a period of time.

For instance, to compare two or more instantaneous measurements, the computing device can compare historic data (i.e., data collected at an earlier time) to more recent data collected. In examples where the sensor 207 includes a pressure sensor, the computing device can compare the interstitial pressure in the historic data from the sensor 207 and determine if there is a change in the interstitial pressure based on the processed data.

Similarly, in examples where the sensor 207 collects data continuously over a period of time, the computing device can process the historical data collected at an earlier time to more recent collected data to determine changes over time. In examples where the sensor 207, in examples where the sensor 207 includes a glucose sensor and the glucose levels were collected over a number of hours or days to determine, the computing device can determine the changes in glucose levels over time. This can help determine, for instance, whether a subject has diabetes. In another example, this can help determine how the subject responds to certain food or drugs, for example. According to another example embodiment, in examples where the sensor 207 collects data related to drug concentrations in a subject’s body, the computing device can process the data to determine drug dosage recommendations. Many example implementations are possible.

Example embodiments shown in FIGS. 2A-2K can help determine a condition of the subject. In some examples, determining a condition of the subject can involve accessing a profile of the subject. For instance, the computing system can include a database storing a number of profiles associated with one or more subjects. Profiles can include information about the subject. For instance, a profile can include, but is not limited to, one or more of the following: (i) breed; (ii) sex; (iii) weight; (iv) age; (v) location; and/or (vi) medical record. In some examples, a subject’s medical record can include, but is not limited to, subject demographic information, vital signs at each clinical visit, diagnoses, medications, treatment plans, progress notes, subject problems, vaccine history, test results, and imaging data, such as radiographs. The demographic data may include species, breed, weight, age, gender, and geographic location, for example. In some examples, the profile of the subject may also include information on test results (for example, complete blood count (CBC), blood chemistry, pathology, urinalysis, serology, and PCR (polymerase chain reaction) panels/assays), vector of exposure, and diagnoses.

In some example implementations, accessing the profile of the subject can include mapping a subject identifier included in the received data to a subject identifier in the profile (e.g., subject’s name and/or numeric, alpha, or alphanumeric code specific to the subject). Other techniques of associating the received data to profile of a subject are possible.

In some examples, if a profile does not exist, or has not been created for a subject, the computing can create and/or prompt a user (e.g., a pet owner) to create a profile of the subject. For instance, a user may receive a message (e.g., text message, e-mail, notification, etc.) prompting a user to create a profile for a subject. In some examples, this message may be sent to a mobile computing device associated with the subject and/or user.

In some examples, determining a condition of the subject can involve applying a machine learning algorithm to the received data and the historic data. As noted above, determining one or more conditions of a subject can be based both on the received data and the historical data (e.g., data collected at an earlier time than the received data). In an example, the machine learning algorithm may determine there is high likelihood that the subject has diabetes based on the detected glucose levels identified in the received data and in the historic data. Many examples are possible.

The machine learning model may be trained using training data that shares a characteristic with a subject to be analyzed by the device 200A and/or device 200B. Training the machine learning model may include inputting one or more training data samples into the machine learning model, predicting, by the machine learning model, an outcome of a determined condition of the one or more training data samples, comparing the at least one outcome to the characteristic of the one or more training samples, and adjusting, based on the comparison, the machine learning model. For example, if a user is attempting to determine whether a subject has diabetes, the machine learning model may be trained by inputting training data of subjects with known diagnoses of diabetes, predicting, by the machine learning model, whether the subject has diabetes, comparing the predicted determination to the known determination, and adjusting, based on the comparison, the machine learning model.

In some examples, the training data may include labeled training data (supervised learning), partially labeled training data (semi-supervised learning), or unlabeled training data (unsupervised learning). In some examples, training may include reinforcement learning.

The machine learning model may include an artificial neural network, a support vector machine, a regression tree, an ensemble of regression trees, or some other machine learning model architecture or combination of architectures.

The training data may include data obtained from tests performed either at laboratories or using instruments at the POC terminal, and clinical history data derived from integrated veterinary clinic practice information management software (PIMS). In some aspects of the disclosure, the data samples are collected over a period of time and stored in the one or more databases.

In some examples, the machine learning model of the computing device may be adjusted based on training such that if the outcome of a determined likelihood matches the likelihood of the training data, the machine learning model is reinforced and if the outcome of a determined likelihood does not match the likelihood of the training data of the training data, the machine learning model is modified. In some examples, modifying the machine learning model includes increasing or decreasing a weight of a factor within the neural network of the machine learning model. In other examples, modifying the machine learning model includes adding or subtracting rules during the training of the machine learning model.

The machine learning algorithm can also access a profile of the subject, which includes information about the subject. For instance, the profile of the subject can include information such as breed, sex, and/or weight of the subject. The profile may additionally or alternatively include information related to the subject’s medical records. The subject’s medical records may include, but is not limited to, demographic information, vital signs at each clinical visit, diagnoses, medications, treatment plans, progress notes, subject problems, vaccine history, test results, and imaging data, such as radiographs.

In some examples, determining one or more conditions of a subject can also involve receiving test results data associated with the subject. In examples, the test result data includes data associated with a biological sample of the subject. For instance, in examples, the biological sample of the subject can include, but is not limited to, one or more of the following: blood, urine, saliva, fecal matter, secretion, excretion, Fine Needle Aspirate (FNA), lavage fluids, body cavity fluids, semen, bacteria, ear wax, skin cells, fecal matter, and biopsied samples. Test may additionally include one or more of the following: blood coagulation test, polymerase chain reaction (PCR) test, and/or immunoassay, among other possibilities.

In example implementations, the test result data may include a marker associated with the condition. For instance, certain levels of glucose can be used to detect or indicate an diabetes in dogs. In some examples, if a test result reaches a threshold level of a marker, the machine learning algorithm may determine that the subject has one or more conditions and/or a high likelihood of one or more conditions. In some example implementations, the computing device may change and/or update the machine learning algorithm based at least in part on whether the test result data includes the marker. For instance, in some examples, modifying the machine learning model can includes increasing or decreasing a weight of a factor, such as the existence and/or level of a marker, within the neural network of the machine learning model. In other examples, modifying the machine learning model includes adding or subtracting rules, such as factoring in the existence and/or level of a marker, during the training of the machine learning model.

Once the computing device has determined one or more conditions of the subject based at least in part on the processed data from device 200A and/or 200B and the historic data from device 200A and/or 200B, the computing device can then provide an indication of one or more conditions of the subject. In example implementations, providing an indication of the one or more conditions can involve transmitting instructions that cause a computing device, such as a smartphone associated with the subject, to display one or more graphical indications of the determined indications of the subject. For instance, the computing device can transmit instructions to provide an indication that there is an increased likelihood that the subject has diabetes based on the processed data indicating glucose levels of collected interstitial fluid and the historic data indicating glucose levels of interstitial fluid collected at an earlier time. In another example, the computing device can transmit instructions to provide an indication that there are no indicators of edema based on the processed data indicating the interstitial pressure and the historic data indicating interstitial pressure. Many example implementations are possible.

Turning to FIG. 3, in some embodiments, the device 300 further comprises a peel-to-expose package with a first tab 302 and a second tab 304. In some examples, first tab 302 and a front side of second tab 304 are configured to attach to one another when in a closed position, such as by an adhesive or other type of fastening mechanism such as a hook and loop fastener or the like. When the first tab 302 is peeled away from the front side of second tab 304, the microneedle array in device 200 is exposed. In the embodiment depicted in FIG. 3, the microneedle array may be coupled to the sample storage layer according to any embodiments depicted in FIGS. 2C-2K, which may in turn be disposed within second tab 304.

In examples, when the first tab 302 is peeled away from the front side of the second tab 304, an adhesive is exposed to adhere the device 300 to the subject. In example implementations the adhesive can help adhere the device 300 to the appendage of the subject over a period of time (e.g., hours, days, or weeks) to allow the sensor 207 to collect data over a period of time. In example implementations, the device 300 may be an ear tag. The adhesive on the outer layer 202 can include strong adhesives to allow the ear tag to remain adhered to the subject’s ear for an extended period of time. In example implementations, the adhesive can include materials such as vinyl resins, acrylates, methacrylates, epoxy diacrylates, acrylic polymer, among other possibilities. Further, the adhesive can be hypoallergenic to prevent irritation.

First tab 302 and second tab 304 may also be configured to re-seal to each other after the sample is collected and stored in the sample storage layer of the device 300. In some embodiments, the first tab 302 may be configured to reseal to removable layer 310. In some embodiments, the first tab 302 may be configured to re-seal to both the second tab 304 and the removable layer 310.

In example embodiments, as described above, the device 300 may utilize an outer layer surrounding one or more microneedles of the microneedle array to provide further structural support to device 300 and/or components thereof.

For example, a back side of second tab 304 containing the sample storage layer of device 300 may be exposed by peeling back a removable layer 310. In embodiments, the second tab 304 is positioned between the removable layer 310 and the first tab 302. The removable layer 310, when closed, may protect a back side of the sample storage layer of device 300. When opened, e.g. at least partially separated from the first tab 302, the sample storage layer of device 300 is exposed from the back side, thus allowing the sample to be extracted from the sample storage layer for testing. In some examples, removable layer 310 may be made of a clear or transparent material to allow a user to see at least a portion of the back side of the sample storage layer of device 300 without removing the removable layer 310. In these embodiments, a user can determine that a sufficient volume of sample has been collected in the sample storage layer of device 300 based on either a change in color of the sample storage layer (e.g., as the sample storage layer is saturated with sample) or another indication by the sample storage layer.

In some embodiments, the device of FIG. 3 optionally may include a pressure indicator in the form of a compressible button 308 configured to provide haptic feedback to a user of the device. When a predetermined pressure is applied to the compressible button 308 by the user, the compressible button 308 may make an audible clicking sound and/or provide haptic feedback to alert the user that the correct pressure is being applied. The compressible button 308, in some examples, may be made of a material and in a shape so that when appropriate pressure is applied and the button is compressed, the sound is made and/or the sample may be visually inspected via removable layer 310, particularly if one or more portion of removable layer 310 comprise a transparent material. Further, in some examples, the compressible button 308 may be positioned in second tab 304 behind the microneedle array of device 300 so that, when the user pushes against it when drawing sample from the animal, the haptic feedback alerts the user that the device is being applied with the appropriate pressure.

### EXAMPLE METHODS AND ASPECTS

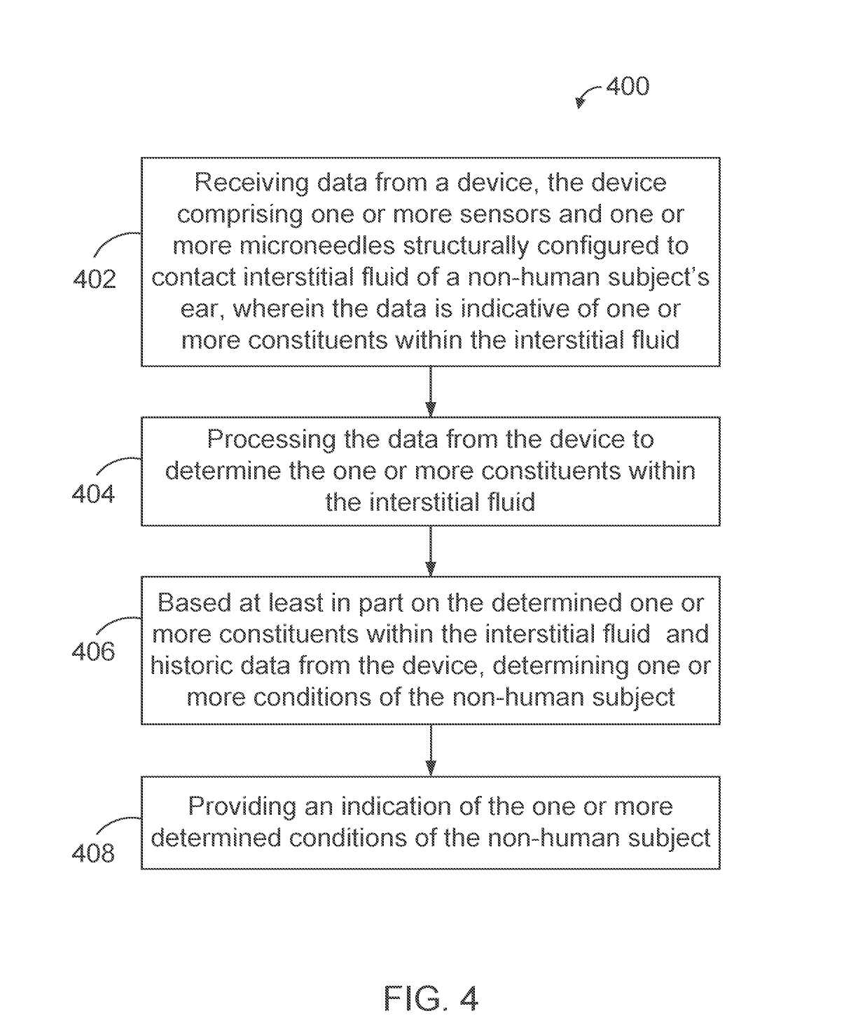

Now referring to FIG. 4, an example computer implemented method for monitoring biometric data from a non-human subject. Method 400 shown in FIG. 4 presents an example computer implemented method for identifying a condition that could be used such as the computing device 100 shown in FIG. 1, for example. Further, devices or systems may be used or configured to perform logical functions presented in FIG. 4. In other examples, components of the devices and/or systems may be arranged to be adapted to, capable of, or suited for performing the functions, such as when operated in a specific manner. Method 400 may include one or more operations, functions, or actions as illustrated by one or more of blocks 402-406. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 402, method 400 involves receiving data a from a device, the device comprising one or more sensors and one or more microneedles structurally configured to contact interstitial fluid of a non-human subject’s ear, wherein the data is indicative of one or more constituents within the interstitial fluid.

In examples, the one or more sensors comprise a pressure sensor, and wherein the data is indicative of a detected interstitial pressure. In these examples, determining the one or more conditions of the non-human subject is based at least in part on the detected interstitial pressure.

In examples, the one or more sensors comprise a glucose sensor, and wherein the data is indicative of a detected glucose level. In these examples, determining the one or more conditions of the non-human subject is based at least in part on the detected glucose level.

In examples, the one or more sensors comprises a salinity sensor, and wherein the data is indicative of a detected salinity level. In these examples, the determining the one or more conditions of the non-human subject is based at least in part on the detected glucose level.

In examples, the one or more sensors is configured to detect a drug concentration within the interstitial fluid.

In examples, the one or more sensors is configured to detect one or more biomarkers in the interstitial fluids. In these examples, the one or more biomarkers includes at least one of hormones, electrolytes, peptides, enzymes, proteins, or antibodies.

At block 404, method 400 involves processing the data from the device to determine the one or more constituents within the interstitial fluid.

At block 406, method 400 involves, based at least in part on the determined one or more constituents within the interstitial fluid and historic data from the device, determining one or more conditions of the non-human subject.

In examples, determining the one or more conditions of the non-human subject comprises applying a machine learning algorithm to the processed data from the device and historic data from the device.

In examples, determining the one or more conditions of the non-human subject is based at least in part on a profile of the non-human subject

At block 408, method 400 involves providing an indication of the one or more determined conditions of the non-human subject.

The singular forms of the articles “a,” “an,” and “the” include plural references unless the context clearly indicates otherwise.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

What is claimed is:

1. A computer implemented method for monitoring biometric data from a non-human subject, the method comprising:
receiving data from a device, the device comprising one or more sensors and one or more microneedles structurally configured to contact interstitial fluid of a non-human subject’s ear, wherein the data is indicative of one or more constituents within the interstitial fluid;
processing the data from the ear tag to determine the one or more constituents within the interstitial fluid;
based at least in part on the determined one or more constituents within the interstitial fluid and historic data from the device, determining one or more conditions of the non-human subject; and
providing an indication of the one or more determined conditions of the non-human subject.

2. The computer implemented method of claim 1, wherein the one or more sensors comprise a pressure sensor, and wherein the data is indicative of a detected interstitial pressure.

3. The computer implemented method of claim 2, wherein determining the one or more conditions of the non-human subject is based at least in part on the detected interstitial pressure.

4. The computer implemented method of claim 1, wherein the one or more sensors comprise a glucose sensor, and wherein the data is indicative of a detected glucose level.

5. The computer implemented method of claim 4, wherein determining the one or more conditions of the non-human subject is based at least in part on the detected glucose level.

6. The computer implemented method of claim 1, wherein the one or more sensors comprises a salinity sensor, and wherein the data is indicative of a detected salinity level.

7. The computer implemented method of claim 6, wherein determining the one or more conditions of the non-human subject is based at least in part on the detected glucose level.

8. The computer implemented method of claim 1, wherein the one or more sensors is configured to detect a drug concentration in the interstitial fluid.

9. The computer implemented method of claim 1, wherein the one or more sensors is configured to detect one or more biomarkers in the interstitial fluids.

10. The computer implemented method of claim 9, wherein the one or more biomarkers includes at least one of hormones, electrolytes, peptides, enzymes, proteins, or antibodies.

11. The computer implemented method of claim 1, wherein determining the one or more conditions of the non-human subject comprises applying a machine learning algorithm to the processed data from the device and historic data from the device.

12. The computer implemented method of claim 1, wherein determining the one or more conditions of the non-human subject is based at least in part on a profile of the non-human subject.

13. A non-transitory computer-readable medium, having stored thereon program instructions that, upon execution by a processor, cause performance of a set of operations comprising:
receiving data from a device, the device comprising one or more sensors and one or more microneedles structurally configured to contact interstitial fluid of a non-human subject’s ear, wherein the data is indicative of one or more constituents within the interstitial fluid;
processing the data from the ear tag to determine the one or more constituents within the interstitial fluid;
based at least in part on the determined one or more constituents within the interstitial fluid and historic data from the device, determining one or more conditions of the non-human subject; and
providing an indication of the one or more determined conditions of the non-human subject.

14. The non-transitory computer-readable medium of claim 13, wherein the one or more sensors comprise a pressure sensor, and wherein the data is indicative of a detected interstitial pressure.

15. The non-transitory computer-readable medium of claim 14, wherein determining the one or more conditions of the non-human subject is based at least in part on the detected interstitial pressure.

16. The non-transitory computer-readable medium of claim 13, wherein the one or more sensors comprise a glucose sensor, and wherein the data is indicative of a detected glucose level.

17. The non-transitory computer-readable medium of claim 16, wherein determining the one or more conditions of the non-human subject is based at least in part on the detected glucose level.

18. The non-transitory computer-readable medium of claim 13, wherein the one or more sensors comprises a salinity sensor, and wherein the data is indicative of a detected salinity level.

19. The non-transitory computer-readable medium of claim 18, wherein determining the one or more conditions of the non-human subject is based at least in part on the detected glucose level.

20. A computing system comprising:
a processor; and
a non-transitory computer-readable medium, having stored thereon program instructions that, upon execution by the processor, cause performance of a set of operations comprising:
receiving data from a device, the device comprising one or more sensors and one or more microneedles structurally configured to contact interstitial fluid of a non-human subject’s ear, wherein the data is indicative of one or more constituents within the interstitial fluid;
processing the data from the device to determine the one or more constituents within the interstitial fluid;
based at least in part on the determined one or more constituents within the interstitial fluid and historic data from the device, determining one or more conditions of the non-human subject; and
providing an indication of the one or more determined conditions of the non-human subject.
